(19) United States
(12) Patent Application Publication
(10) Pub. No.: US 2025/0143565 A1
(43) Pub. Date: May 8, 2025

(54) VISUALIZATION OF OCULAR LENS BASED ON TILTED OCT IMAGING

(71) Applicant: Alcon Inc., Fribourg (CH)

(72) Inventors: Chad P. Byers, Mission Viejo, CA (US); Mark Andrew Zielke, Lake Forest, CA (US); Christopher Sean Mudd, Lake Forest, CA (US)

(21) Appl. No.: 18/934,491

(22) Filed: Nov. 1, 2024

Related U.S. Application Data
(60) Provisional application No. 63/596,055, filed on Nov. 3, 2023.

(51) Int. Cl. A61B 3/10; A61B 3/00; G16H 30/40; G16H 40/63

(52) U.S. Cl. CPC A61B 3/102; A61B 3/0025; G16H 30/40; G16H 40/63

(57) ABSTRACT

A system and method for visualizing an eye using an optical coherence tomography (“OCT”) device includes a controller having a processor and a tangible, non-transitory memory on which instructions are recorded. The OCT device produces an OCT beam defined by an OCT beam axis. The controller is adapted to receive a first dataset captured with the OCT beam axis at a first tilt angle from a first visual axis. The controller is adapted to receive a second dataset captured with the OCT beam axis at a second tilt angle from a second visual axis. A plurality of lens segments is generated based on the first dataset and the second dataset. The controller is adapted to generate a lens profile based in part on the plurality of lens segments.

### PRIORITY CLAIM

This application claims the benefit of priority of U.S. Provisional Patent Application Ser. No. 63/596,055, titled “VISUALIZATION OF OCULAR LENS BASED ON TILTED OCT IMAGING” filed on Nov. 3, 2023, whose inventors are Chad P. Byers, Mark Andrew Zielke, and Christopher Sean Mudd, all of which are hereby incorporated by reference in their entirety as though fully and completely set forth herein.

### INTRODUCTION

The disclosure relates generally to system and method for visualizing an eye using an optical coherence tomography (“OCT”) device. More particularly, the disclosure relates to visualization of the ocular lens using tilted OCT imaging. OCT is a noninvasive imaging technology using low-coherence interferometry to generate high-resolution images of ocular structure. OCT imaging functions partly by measuring the echo time delay and magnitude of backscattered light. Images generated by OCT are useful for many purposes, such as identification and assessment of ocular diseases. OCT images are frequently taken prior to cataract surgery, where an intraocular lens is implanted into a patient's eye. An inherent limitation of OCT imaging is that the illuminating beam cannot penetrate across the iris. Hence posterior regions of the eye, such as the crystalline lens structure behind the iris, may not be properly visualized.

### SUMMARY

Disclosed herein is a system and method for visualizing an eye using an optical coherence tomography (“OCT” hereinafter) device. The system includes a controller having a processor and a tangible, non-transitory memory on which instructions are recorded. The OCT device produces an OCT beam defined by an OCT beam axis. The controller is adapted to receive a first dataset captured with the OCT beam axis at a first tilt angle from a first visual axis. The controller is adapted to receive a second dataset captured with the OCT beam axis at a second tilt angle from a second visual axis. A plurality of lens segments is generated based on the first dataset and the second dataset. The controller is adapted to generate a lens profile based in part on the plurality of lens segments.

The controller may be adapted to perform redundant surface mapping of the plurality of lens segments to generate the lens profile. In some embodiments, the first dataset is captured with the eye focused on a first side and the OCT beam is directed from a temporal region adjacent to the eye on a second side. The first dataset may include volumetric data captured as the OCT beam is rotated around the first visual axis while maintaining a magnitude of the first tilt angle.

In some embodiments, the second dataset is captured with the eye focused along a third side and the OCT beam is directed from a nasal region adjacent to the eye. The second dataset may include volumetric data captured as the OCT beam is rotated around the second visual axis while maintaining a magnitude of the second tilt angle. In some embodiments, the first tilt angle and the second tilt angle are each between about 25 degrees and about 45 degrees. The first tilt angle and the second tilt angle may be each between about 30 degrees and about 35 degrees.

In some embodiments, the first dataset and the second dataset are respectively captured when a pupil of the eye is naturally dilated. In some embodiments, the first dataset and the second dataset are captured when a pupil of the eye is chemically dilated. The controller may be adapted to adjust a longitudinal axis of the lens profile to match a predefined reference axis. The controller may be further adapted to generate first and second corner portions of the lens profile. The first and second corner portions of the lens profile may be generated using an artificial neural network selectively executable by the controller.

Disclosed herein is a method visualizing an eye using an optical coherence tomography (“OCT”) device with a system having a controller with at least one processor and at least one non-transitory, tangible memory. The method includes receiving a first dataset captured with an OCT beam axis at a first tilt angle from a first visual axis, the OCT device producing an OCT beam defined by the OCT beam axis. The method includes receiving a second dataset captured with the OCT beam axis at a second tilt angle from a second visual axis and generating a plurality of lens segments based on the first dataset and the second dataset. The method includes generating a lens profile based in part on the plurality of lens segments.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 is a schematic illustration of a system for visualizing a target site using OCT, the system having a controller;

FIG. 2 is a schematic diagram of a ray-tracing map illustrating the effect of tilted OCT imaging;

FIG. 3 is a schematic flowchart for a method executable by the controller of FIG. 1;

FIG. 4A is a schematic diagram of an example OCT image captured with the OCT beam axis at a first tilt angle from a first visual axis of the eye;

FIG. 4B is a schematic illustration of a subject set up with the OCT beam orientation of FIG. 4A;

FIG. 5A is a schematic diagram of another example OCT image captured with the OCT beam axis at a second tilt angle from a second visual axis of the eye;

FIG. 5B is a schematic illustration of a subject set up with the OCT beam orientation of FIG. 5A;

FIG. 6 is a schematic illustration of overlaid lens segments; and

FIG. 7 is a schematic diagram of a full lens profile generated by the system of FIG. 1.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover modifications, equivalents, combinations, sub-combinations, permutations, groupings, and alternatives falling within the scope of this disclosure as encompassed, for instance, by the appended claims.

### DETAILED DESCRIPTION

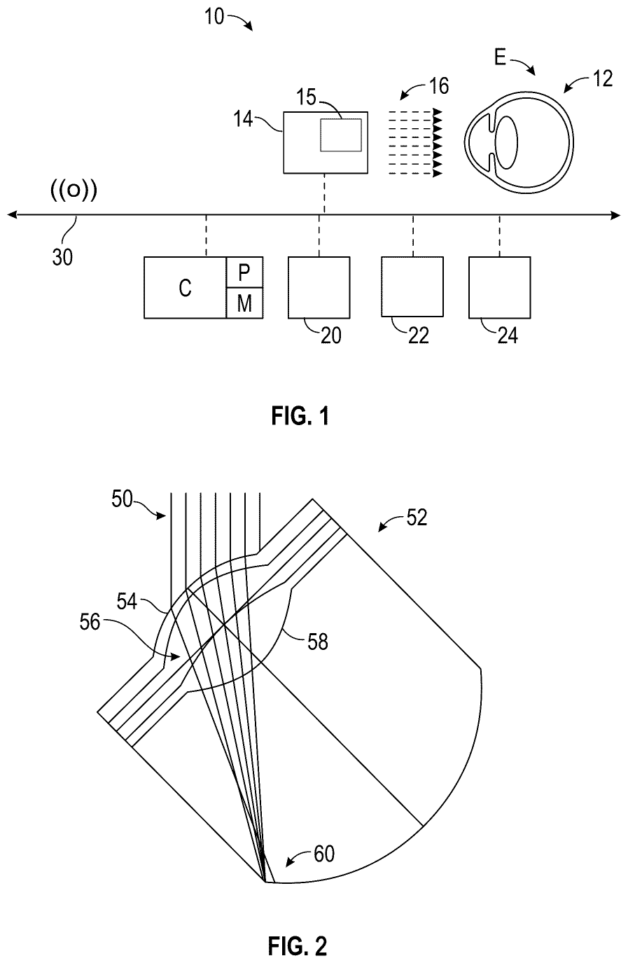

Referring to the drawings, wherein like reference numbers refer to like components, FIG. 1 schematically illustrates a system 10 for visualizing a target site 12 with data captured via an optical coherence tomography image (“OCT” hereinafter) device 14. In the embodiment shown, the target site 12 is an eye E. The OCT device 14 may employ an array of laser beams 16 for illuminating the eye E, with the array of laser beams 16 covering the span of the eye E. In one example, the OCT device 14 is an anterior segment high definition OCT imaging device. The OCT device 14 may employ swept-source OCT. It is to be understood that the OCT device 14 may take many different forms and include multiple and/or alternate components.

Prior to cataract surgery, ophthalmic surgeons make use of a wide variety of algorithms to plan for intraocular lens replacement in order to best correct vision. Biometric measurements of the eye provide data input to these algorithms, such as lens thickness, axial length, anterior chamber depth, etc. In many cases, models are used to infer parameters, such as the equatorial plane position and lens diameter, from observables. However, these inferred parameters may introduce errors to the algorithm.

As described below, the system 10 makes use of volumetric OCT to determine the morphology of the natural lens of the eye E or its replacement, an intraocular lens. The OCT device 14 may be capable of 3D volume scanning produced at a variety of angles to generate near-total coverage of the lens. By capturing OCT volumetric data at multiple angles, redundant surface mapping at the anterior and posterior central regions ensures higher certainty of total lens morphology.

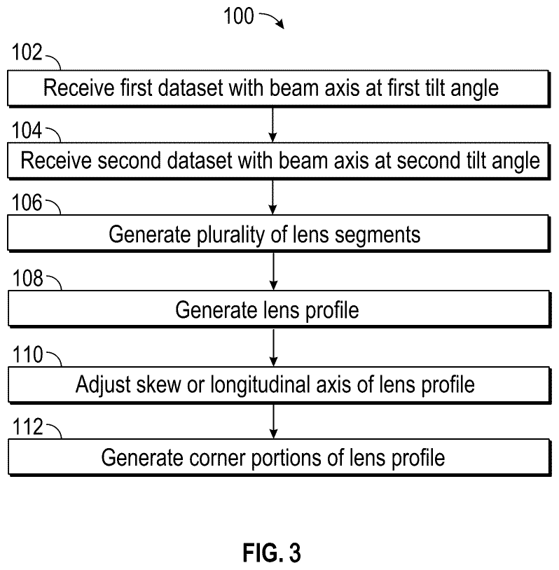

Referring to FIG. 1, the system 10 includes a controller C having at least one processor P and at least one memory M (or non-transitory, tangible computer readable storage medium) on which instructions are recorded for executing method 100 for visualizing a target site 12 using the OCT device 14. Method 100 is shown in and described below with reference to FIG. 3.

The system 10 (via execution of method 100) significantly increases the extent of lens biometric measurements with the use of tilted OCT imaging. Referring to FIG. 2, a schematic diagram illustrating the effect of tilted OCT imaging is shown. FIG. 2 shows an OCT beam 50 passing through an eye model 52 having a sclera 54, pupil 56 and lens 58. The OCT beam 50 is incident on the model eye 52 at an angle of incidence that is about 45 degrees. The ray-tracing simulations here were carried out using an eye model 52 with the optical properties of an average human eye both in dimension and refraction properties. As shown in FIG. 2, the tilted configuration allows for greater beam coverage of the anterior segment of the model eye 52, including the lens 58, as well as the posterior segment of the model eye 52 (c.g., the retina 60). By changing the orientation of the OCT beam 50 (relative to the eye) to the opposite side by 45 degrees (e.g., left), the opposite side of the lens 58 may be imaged fully. Coverage of the lens 58 is further improved by capturing the data when the pupil 56 is dilated. This may be accomplished by either placing the subject in a low-light environment or by administering mydriatics or medicine that make the pupil of the eye dilate. In this example, the pupil 56 is naturally dilated to about 7 mm.

Referring to FIG. 1, the controller C may be configured to receive and transmit data through a user interface 20. The user interface 20 may be installed on a smartphone, laptop, tablet, desktop or other electronic device and may include a touch screen interface or I/O device such as a keyboard or mouse. The user interface 20 may be a mobile application. The circuitry and components of a mobile application (“apps”) available to those skilled in the art may be employed. The user interface 20 may include an integrated processor and integrated memory. The controller C may selectively execute an artificial neural network 22, described below.

The various components of the system 10 of FIG. 1 may communicate via a network 30. The network 30 may be a bus implemented in various ways, such as for example, a serial communication bus in the form of a local area network. The local area network may include, but is not limited to, a Controller Area Network (CAN), a Controller Area Network with Flexible Data Rate (CAN-FD), Ethernet, blue tooth, WIFI and other forms of data connection. The network 30 may be a Wireless Local Area Network (LAN) which links multiple devices using a wireless distribution method, a Wireless Metropolitan Area Networks (MAN) which connects several wireless LANs or a Wireless Wide Area Network (WAN). Other types of connections may be employed.

Referring now to FIG. 3, a flow chart of method 100 executable by the controller C of FIG. 1 is shown. Method 100 need not be applied in the specific order recited herein and some blocks may be omitted. The memory M can store controller-executable instruction sets, and the processor P can execute the controller-executable instruction sets stored in the memory M.

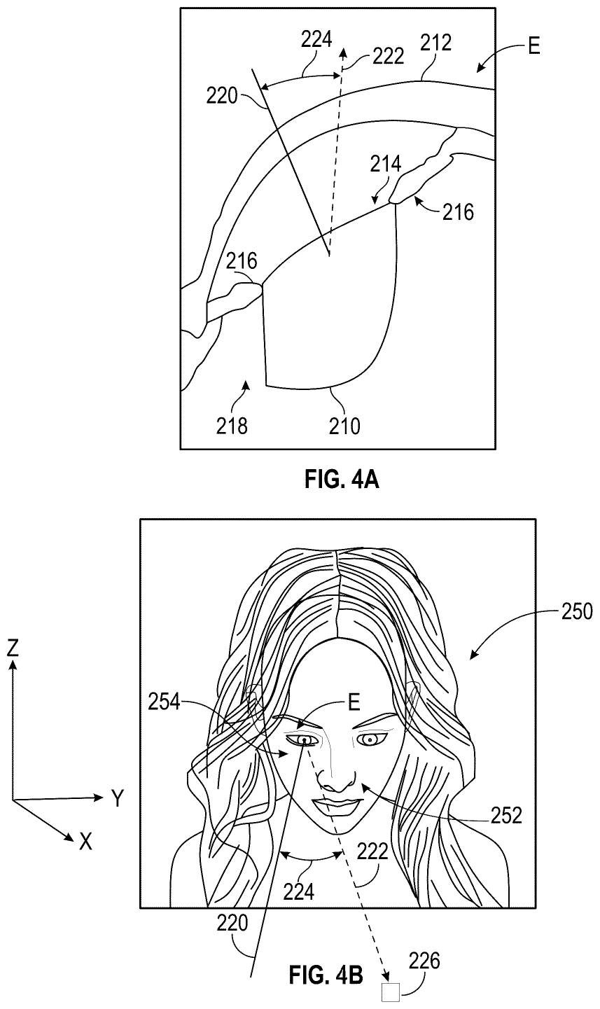

Per block 102 of FIG. 3, the controller C is configured to receive a first dataset captured by the OCT device 14. FIG. 4A is a schematic diagram of an example OCT image (B-scan) captured of an eye E. Shown in FIG. 4A are the lens 210, sclera 212, pupil 214 and iris 216. The controller C is adapted to reconstructed the peripheral portion 218 of the lens 210 that is behind the iris 216.

FIG. 4B shows a subject 250 that is set up with the beam orientation of FIG. 4A. Referring to FIGS. 4A and 4B, the first dataset is captured with an OCT beam axis 220 at a first tilt angle 224 from a first visual axis 222. The OCT device 14 produces an OCT beam defined by an OCT beam axis. The OCT beam axis 220 is the travel direction of the light source (e.g., laser beam) emanating from the OCT device 14. A single scan directed at a spot on the eye results in a depth scan of the structure of the physical sample into which the OCT beam is directed, along the incident direction. A depth scan may be referred to as an “A-scan” and is configured to scan to a detected depth along the OCT beam axis, or the travel direction of the of the OCT device. The OCT beam may be moved in a continual manner about the eye E using a steering unit 15 within the OCT device 14, thereby enabling multiple depth scans along a transverse scan range. Such a line of A-scans may be referred to as a B-scan or row scan. Volumetric OCT imaging provides additional data, as a 3-dimensional manifold may be fitted to the anterior and posterior lens surfaces.

The visual axis 222 of the eye E may be defined to extend from a physical point in the eye (such as the fovea) to a fixation point 226 that the subject 250 is directed or looking towards. The visual axis 222 may shift, or reorient, depending on the orientation of the subject 250. The fixation point 226 may be an object or an imaginary point. It is to be understood that the tilt angles may also be accomplished without the use of a fixation point or target. In some embodiments, a slit lamp microscope may be employed to measure the visual axis of the eye. A slit lamp generally has a high intensity light source that is adapted to focus and shine the light as a slit, allowing an operator to view parts of the eye in greater detail (relative to the naked eye).

In the embodiment shown in FIG. 4B, the first dataset is captured with the eye focused on a first side (e.g., the eye focused towards the nose 252) and the OCT beam (represented by the OCT beam axis 220) is directed from a temporal region 254 adjacent to the eye E on the second side (opposite to the first side). For example, the first dataset may be captured as the right eye looks towards the left and the OCT beam is incoming from the right temporal region. It is understood that the orientation of the subject 250 and OCT beam may be varied based on the application at hand. The first dataset may include volumetric data captured as the OCT beam is rotated around the first visual axis 222 while maintaining a magnitude of the first tilt angle 224 (e.g., defining a virtual cone around the first visual axis 222). The OCT device 14 may incorporate swept-source optical coherence tomography to obtain a 3D volume scan produced at a variety of angles and generate near-total coverage of the lens.

In the example OCT image shown in FIG. 4A, dilation of the pupil 214 was accomplished naturally under low ambient light conditions. For example, the head of the subject 250 may be placed under a dark veil to induce natural dilation. As an added benefit, collecting pre-operative subject data under low ambient light conditions accurately identifies the vertical axis of the eye E for subsequent tracking and reference without sacrificing visual acuity for the physician.

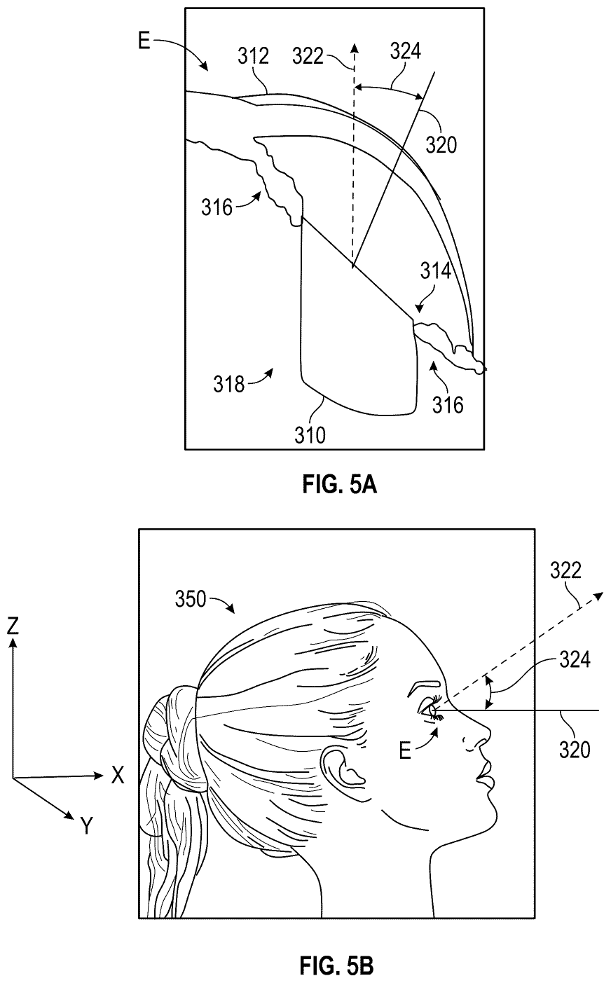

Per block 104 of FIG. 3, the controller C is configured to receive a second dataset captured with the OCT device 14, an example of which is shown in FIG. 5A. FIG. 5A shows an OCT image (B-scan) captured of the eye E with lens 310, sclera 312, pupil 314 and iris 316. FIG. 5B shows a subject 350 that is set up with the beam orientation of FIG. 5A. Referring to FIGS. 5A and 5B, the second dataset is captured with the OCT beam axis 320 at a second tilt angle 324 from a second visual axis 322. The OCT beam axis 320 is the travel direction of the light source (e.g., laser beam) emanating from the OCT device 14. Referring to FIG. 5A, the controller C is adapted to reconstructed the peripheral portion 318 of the lens 310 that is behind the iris 316.

Referring to FIG. 5B, the second dataset may be captured with the eye focused along a third side (e.g., in a superior direction or along the Z axis) and the OCT beam (represented by the OCT beam axis 320) is incoming from a nasal region under the eye E. It is understood that the orientation of the subject 350 and OCT beam may be varied based on the application at hand. The second dataset may include volumetric data captured as the OCT beam is rotated around the second visual axis 322 while maintaining the second tilt angle 324 (e.g., defining a virtual cone around the second visual axis 322). In some embodiments, the first tilt angle and the second tilt angle are each between about 30 degrees and about 35 degrees. In other embodiments, the first tilt angle 224 and the second tilt angle 324 are each between about 25 degrees and about 45 degrees.

The first dataset and the second dataset may be captured when the pupil 214, 314 is naturally dilated. The first dataset and the second dataset may be captured when the pupil 214, 314 is chemically dilated. In some embodiments, the first dataset and the second dataset may be captured when the pupil 214, 314 is not dilated.

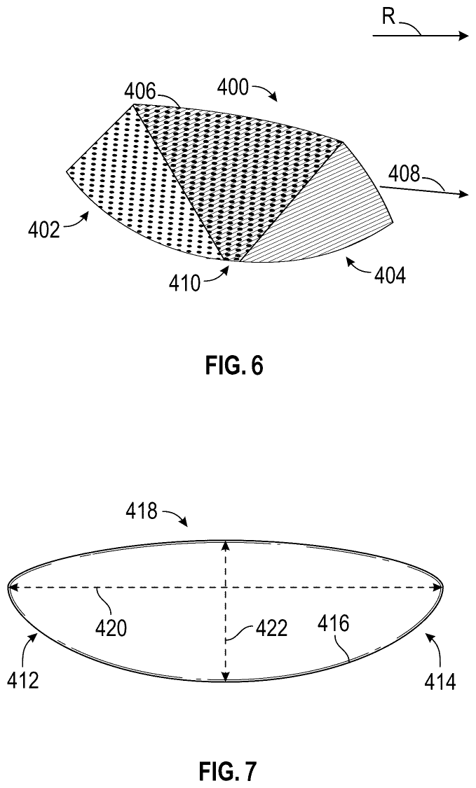

Per block 106 of FIG. 3, the controller C is configured to generate a plurality of lens segments 400, such as first segment 402 and second segment 404 shown in FIG. 6, based on the first dataset and the second dataset. The first segment 402 is shown stippled and the second segment 404 is shown hatched in FIG. 6. The manually segmented overlays indicate refraction-uncorrected lens visibility at an anterior central region and a posterior central region.

Per block 108 of FIG. 3, the method 100 includes generating a lens profile 410 based in part on the plurality of lens segments 400. The controller C is adapted to perform redundant surface mapping of the lens segments 400, to produce multiple overlapping zones, such as overlapping zone 406 in FIG. 6. The multiple overlapping zones enable a robust determination of the lens profile 410.

Per block 110, the controller C may be configured to adjust any tilt or skew of the lens profile 410, such as by adjusting a longitudinal axis 408 of the lens profile 410 relative to a predefined reference axis R, shown in FIG. 6. Advancing to block 112, the controller C may be configured to generate the peripheral portions of the lens, such as first and second corner portions 412, 414 shown in FIG. 7, and obtain a full lens capsule profile 416 of a lens 418.

Reconstruction of the peripheral portion of the lens may be accomplished using one or more machine learning models, such as an artificial neural network 22 (see FIG. 1). The adjustment of any tilt or skew of the lens profile 410 (relative to a reference axis R) may also be done through neural network 22.

The neural network 22 is trained using training datasets and is selectively executable by the controller C. The training process occurs in a closed loop or iterative fashion, with the neural network 22 being trained until a certain criteria is met, i.e., until the discrepancy between the network outcome and ground truth reaches a point below a certain threshold. As a predefined loss function related to the training dataset is minimized, the neural network 22 reaches convergence. The convergence signals the completion of the training.

The system 10 may be configured to be “adaptive” and updated periodically after the collection of additional training data for the artificial neural network 22. It is to be understood that the system 10 is not limited to a specific neural network methodology and the reconstruction of missing information from the lens profile may be assisted by other deep neural network methodologies available to those skilled in the art.

A full image of the preoperative crystalline lens structure is useful for selecting an appropriate power for the intraocular lens during pre-operative assessments for cataract surgery. The controller C is configured to obtain at least one lens parameter based on the full lens capsule profile 416. Referring to FIG. 7, the lens parameters may include the lens diameter 420 and thickness 422 of the lens 418 along the lens diameter 420. The lens parameters may be outputted to a lens selection module 24 (see FIG. 1) for selecting an intraocular lens (not shown) for implantation into the eye E. This information is particularly useful for intraocular lenses that are accommodative in nature, as their functional performance has been observed to be correlated to the lens diameter 420.

The system 10 may be used to accomplish early screening for cortical cataracts. Because cortical cataracts often begin formation on the edges of the lens, full lens capsule profile 416 allows clinicians to visualize early-stage cortical cataracts.

In summary, the system 10 illustrates a robust way to capture greater information using an OCT device 14. The system 10 enables improvements in the surgical planning process for cataract surgery, including OCT-based cataract grading and planning. By inspecting the scattering properties of a large portion of the lens, the surgeon may glean information regarding the structure and degree of difficulty of the surgery, allowing improved planning. Following lens-replacement surgery, the system 10 provides advantages in imaging peripheral features of intra-ocular lenses, such as haptic seating. The method 100 provides benefits in the planning of post implant interventions.

The controller C of FIG. 1 includes a computer-readable medium (also referred to as a processor-readable medium), including a non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random-access memory (DRAM), which may constitute a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, other magnetic medium, a CD-ROM, DVD, other optical medium, a physical medium, a RAM, a PROM, an EPROM, a FLASH-EEPROM, other memory chip or cartridge, or other medium from which a computer can read.

Look-up tables, databases, data repositories or other data stores described herein may include various kinds of mechanisms for storing, accessing, and retrieving various kinds of data, including a hierarchical database, a set of files in a file storage system, an application database in a proprietary format, a relational database energy management system (RDBMS), etc. Each such data store may be included within a computing device employing a computer operating system such as one of those mentioned above and may be accessed via a network in one or more of a variety of manners. A file system may be accessible from a computer operating system and may include files stored in various formats. An RDBMS may employ the Structured Query Language (SQL) in addition to a language for creating, storing, editing, and executing stored procedures, such as the PL/SQL language mentioned above.

The flowchart shown in the FIGS. illustrates an architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by specific purpose hardware-based systems that perform the specified functions or acts, or combinations of specific purpose hardware and computer instructions. These computer program instructions may also be stored in a computer-readable medium that can direct a controller or other programmable data processing apparatus to function in a particular manner, such that the instructions stored in the computer-readable medium produce an article of manufacture including instructions to implement the function/act specified in the flowchart and/or block diagram blocks.

The numerical values of orders (e.g., of quantities or conditions) in this specification, including the appended claims, are to be understood as being modified in each respective instance by the term “about” whether or not “about” actually appears before the numerical value. “About” indicates that the stated numerical value allows some slight imprecision (with some approach to exactness in the value; about or reasonably close to the value; nearly). If the imprecision provided by “about” is not otherwise understood in the art with this ordinary meaning, then “about” as used herein indicates at least variations that may arise from ordinary methods of measuring and using such orders. In addition, disclosure of ranges includes disclosure of each value and further divided ranges within the entire range. Each value within a range and the endpoints of a range are hereby disclosed as separate embodiments.

The detailed description and the drawings or FIGS. are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A system of visualizing an eye using an optical coherence tomography (“OCT”) device, the system comprising:
a controller having at least one processor and at least one non-transitory, tangible memory on which instructions are recorded;
wherein the OCT device produces an OCT beam defined by an OCT beam axis, execution of the instructions by the processor causing the controller to:
receive a first dataset captured with the OCT beam axis at a first tilt angle from a first visual axis of the eye;
receive a second dataset captured with the OCT beam axis at a second tilt angle from a second visual axis of the eye;
generate a plurality of lens segments based on the first dataset and the second dataset; and
generate a lens profile based in part on the plurality of lens segments.

2. The system of claim 1, wherein the controller is adapted to perform redundant surface mapping of the plurality of lens segments to generate the lens profile.

3. The system of claim 1, wherein the first dataset is captured with the eye focused on a first side and the OCT beam is directed from a temporal region adjacent to the eye on a second side.

4. The system of claim 3, wherein the first dataset includes volumetric data captured as the OCT beam is rotated around the first visual axis while maintaining a magnitude of the first tilt angle.

5. The system of claim 3, wherein the second dataset is captured with the eye focused along a third side and the OCT beam is directed from a nasal region adjacent to the eye.

6. The system of claim 5, wherein the second dataset includes volumetric data captured as the OCT beam is rotated around the second visual axis while maintaining a magnitude of the second tilt angle.

7. The system of claim 1, wherein the first tilt angle and the second tilt angle are each between about 25 degrees and about 45 degrees.

8. The system of claim 1, wherein the first tilt angle and the second tilt angle are each between about 30 degrees and about 35 degrees.

9. The system of claim 1, wherein the first dataset and the second dataset are respectively captured when a pupil of the eye is naturally dilated.

10. The system of claim 1, wherein the first dataset and the second dataset are captured when a pupil of the eye is chemically dilated.

11. The system of claim 1, wherein the controller is adapted to adjust a longitudinal axis of the lens profile to match a predefined reference axis.

12. The system of claim 1, wherein the controller is further adapted to generate first and second corner portions of the lens profile.

13. The system of claim 12, wherein the first and second corner portions of the lens profile are generated using an artificial neural network selectively executable by the controller.

14. A method visualizing an eye using an optical coherence tomography (“OCT”) device with a system having a controller with at least one processor and at least one non-transitory, tangible memory, the method comprising:
receiving a first dataset captured with an OCT beam axis at a first tilt angle from a first visual axis, the OCT device producing an OCT beam defined by the OCT beam axis;
receiving a second dataset captured with the OCT beam axis at a second tilt angle from a second visual axis;
generating a plurality of lens segments based on the first dataset and the second dataset; and
generating a lens profile based in part on the plurality of lens segments.

15. The method of claim 14, further comprising:
performing redundant surface mapping of the plurality of lens segments to generate the lens profile.

16. The method of claim 14, further comprising:
capturing the first dataset when the eye is focused on a first side and the OCT beam is directed from a temporal region adjacent to the eye on a second side, the first dataset including volumetric data captured as the OCT beam axis is rotated around the first visual axis.

17. The method of claim 16, further comprising:
capturing the second dataset when the eye is focused along a third side and the OCT beam is directed from a nasal region adjacent to the eye, the second dataset including the volumetric data captured as the OCT beam axis is rotated around the second visual axis. 18 The method of claim 14, further comprising:
selecting the first tilt angle and the second tilt angle to be between about 25 degrees and about 45 degrees; and
capturing the first dataset and the second dataset respectively when a pupil of the eye is dilated.

19. The method of claim 14, further comprising:
adjusting a longitudinal axis of the lens profile to match a predefined reference axis; and
generating first and second corner portions of the lens profile using an artificial neural network selectively executable by the controller.

20. A system of visualizing an eye using an optical coherence tomography (“OCT”) device, the system comprising:
a controller having at least one processor and at least one non-transitory, tangible memory on which instructions are recorded;
wherein the OCT device produces an OCT beam defined by an OCT beam axis, execution of the instructions by the processor causing the controller to:
receive a first dataset captured with the OCT beam axis at a first tilt angle from a first visual axis of the eye;
receive a second dataset captured with the OCT beam axis at a second tilt angle from a second visual axis of the eye;
generate a plurality of lens segments based on the first dataset and the second dataset; and
perform redundant surface mapping of the plurality of lens segments and generate a lens profile based in part on the plurality of lens segments;
wherein the first dataset is captured with the eye focused on a first side and the OCT beam is directed from a temporal region adjacent to the eye on a second side;
wherein the second dataset is captured with the eye focused along a third side and the OCT beam is directed from a nasal region adjacent to the eye; and
wherein the first tilt angle and the second tilt angle are each between about 25 degrees and about 45 degrees.
